(19) United States
(12) Patent Application Publication
(10) Pub. No.: US 2005/0182297 A1
(43) Pub. Date: Aug. 18, 2005

(54) Imaging scope

(76) Inventors: Dietrich Gravenstein, Gainesville, FL (US); Samsun Lampotang, Gainesville, FL (US); Richard J. Melker, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/982,154

(22) Filed: Nov. 5, 2004

Related U.S. Application Data
(63) Continuation-in-part of application No. 10/807,965, filed on Mar. 24, 2004.
Application No. 10/807,965 is a continuation-in-part of application No. 10/001,560, filed on Oct. 23, 2001, now abandoned.
Application No. 10/001,560 is a continuation of application No. 09/007,939, filed on Jan. 16, 1998, now Pat. No. 6,322,498.
Application No. 09/007,939 is a continuation-in-part of application No. PCT/US97/17954, filed on Oct. 6, 1997.
Application No. PCT/US97/17954 is a continuation of application No. 08/725,779, filed on Oct. 4, 1996, now Pat. No. 6,115,523.

(51) Int. Cl. A61B 1/00; A62B 9/06

(52) U.S. Cl. 600/139

(57) ABSTRACT

Disclosed is an intubation imaging stylet for intubating a patient by use in a tube/imaging stylet combination, said imaging stylet comprising: a malleable stylet having a longitudinal axis and a proximal end and a distal end; a flexible image guide having a longitudinal axis and a proximal end and a distal end, said image guide being connected to said stylet such that a portion of said image guide runs parallel to a portion of said stylet along the longitudinal axis of said stylet and such that the distal end of said image guide is co-extensive with the distal end of said stylet; and at least one flexible illumination fiber having a proximal end and a distal end, said illumination fiber being connected to said stylet such that a portion of said illumination fiber runs parallel to a portion of said stylet along the longitudinal axis of said stylet and such that the distal end of said illumination fiber is co-extensive with the distal end of said stylet; such that in use, said imaging stylet is disposed within a tube for intubating a patient thereby forming an imaging stylet/tube combination which in use is held by gripping the tube in a pen-like fashion. The imaging stylet/tube combination is thus in use held in one hand, freeing the other hand of the user for other tasks if necessary, as well as permitting intubation in the conventional manner. To facilitate this, the center of gravity of the imaging stylet/tube combination is located in essentially the same location along the tube as with a conventional stylet/tube combination.

### CROSS-REFERENCE TO RELATED APPLICATION

This application is a continuation-in-part of co-pending patent application Ser. No. 10/807,965, filed Mar.24, 2004, which is a continuation-in-part of Ser. No. 10/001,560, filed Oct. 23, 2001, now abandoned, which is a continuation of patent application Ser. No. 09/007,939, filed Jan. 16, 1998 (now U.S. Pat. No. 6,322,498; issued Nov. 27, 2001) which is a continuation-in-part of international patent application Ser. No. PCT/US97/17954, filed Oct. 6, 1997; which is a continuation of patent application Ser. No. 08/725,779 filed Oct. 4, 1996 (now U.S. Pat. No. 6,115,523; issued Sep. 5, 2000).

### BACKGROUND OF THE INVENTION

This invention relates to fiber optic imaging scopes and, in a particular embodiment, to intubation scopes. The subject intubation scopes can incorporate a malleable stylet, which can retain its shape when bent, to facilitate intubation. Advantageously, this invention can utilize plastic optical fibers to enhance the safety and efficacy of procedures performed with these novel fiber optic scopes, while simultaneously reducing the costs. Additionally, the scopes of the subject invention can, optionally, utilize a sheath which can reduce the amount of cleaning, i.e., sterilization, required between uses and thereby reduce the costs of use.

It is frequently necessary in medical procedures to insert an endotracheal tube into the trachea of a patient for the purpose of performing diagnostic tests or for the introduction of some means of ventilation, oxygenation, and/or airway protection. Even in the best situations, intubation is often difficult and can give rise to complications. In many patients, establishment of the airway is particularly difficult due to morphologic anomalies such as a large tongue, excessive pharyngeal or laryngeal soft tissue, or tracheal displacement, as well as physiologic events such as laryngospasm, regurgitation of gastric materials, bleeding, or foreign bodies aspiration. These morphologic anomalies and/or events make it difficult to visualize the posterior pharyngeal area and larynx with conventional laryngoscopic maneuvers. In emergency situations, attempts to intubate such patients are difficult and time consuming. Inability to expeditiously intubate the patient and protect the airway can lead to significant hypoxemia, myocardial ischemia, and brain injury. Cases of death have also been related to complications caused by the inability to quickly and clearly see the larynx and trachea.

Proper intubation requires positioning the tip of the tracheal tube within the trachea, midway between the patient's vocal cords and carina. Direct laryngoscopy in many instances is sufficient to intubate the patient, but does not permit the precise confirmation of tip location or tracheal inspection.

If the tracheal tube is not inserted far enough past the vocal cords, the tube may become dislodged and prove to be ineffective in supporting adequate artificial ventilation. Further, the tube may inadvertently end up in the esophagus. Esophageal intubations, resulting from either dislodgement or incorrect initial placement have led to severe morbidity and even death. At the other extreme, if inserted too far and beyond the carina, the tube may only permit ventilation of one lung (as opposed to both lungs). Thus, correct tube placement is essential in order to properly ventilate the patient.

Even the most skilled anesthesiologist may encounter what is commonly referred to as a “difficult” airway. This occurs in about 5% of all operating room intubations, with an even higher incidence of an inability to fully visualize the glottic opening. The incidence level is significantly higher in other areas of the hospital and prehospital environment. Although presurgical examination of the jaw, teeth, mouth opening and neck motion assists in gauging the degree of difficulty likely to be encountered at intubation, not all difficult intubations can be identified in advance. There is always the unexpected difficult airway, discovered only at the time of intubation. In emergency situations, there is little if any time to perform an airway assessment prior to attempting intubation. Thus, all emergency intubations are considered “difficult” intubations.

There are a number of techniques used to assist in difficult intubations. These include laryngoscopy, with or without axial cervical stabilization, fiberoptic bronchoscopy, with or without a transtracheal retrograde wire guide, blind nasal and the lighted stylet techniques.

Fiberoptic bronchoscopy is considered by many as the “gold standard” for viewing the airway and properly positioning a tracheal tube. The complexity of operating and cost of buying, maintaining, cleaning, and repairing existing glass fiberoptic systems, which are fragile, are major factors preventing greater usage of bronchoscopy.

The retrograde wire technique involves placing a needle into the cricothyroid space and advancing a guide wire through the needle and upward through the glottic opening between the vocal cords and pharynx until it emerges from the nose or mouth. After the wire is localized, a fiberoptic bronchoscope or tracheal tube is advanced over the wire into the larynx. This technique is not recommended in emergency situations. Major negative concerns associated with this technique are its invasive nature and the risk for bleeding and infection in the trachea. The wire can also cause injury to the tracheal tissue and/or vocal cords.

A lighted stylet is essentially a standard stylet with a bright light at the distal end. This technique provides only indirect transcutaneous illumination of the trachea. Direct visualization is not possible when using a lighted stylet.

Fiberoptic intubating scopes with cameras and/or eyepieces for viewing that which is illuminated by the fiber optic system have previously been described. See, for example, U.S. Pat. Nos. 3,776,222; 4,742,819; 4,846,153 and 5,363,838. Current fiber optic scopes, for example, intubation scopes and associated systems for imaging the human airways, typically use glass optical fibers. Unfortunately, these intubation scopes and associated systems utilizing glass optical fibers are expensive to purchase, clean, and store. Additionally, the glass optical fibers within these scopes are prone to breaking thereby shortening the life of the scopes.

### BRIEF DESCRIPTION OF THE INVENTION

The subject invention pertains to a system for imaging the human airway having highly advantageous optical, mechanical, ergonomical and physical characteristics. The subject system allows for a user to utilize conventional techniques for the insertion of an endotracheal tube while using the subject imaging scope. The excellent characteristics of the imaging system of the subject invention result, in part, from the use of plastic optical fibers. Plastic optical fibers are more robust than the glass optical fibers used in currently available imaging systems, and are therefore capable of being bent and/or twisted with virtually no concern of breakage. In addition, the lower costs of plastic optical fibers enables scopes of the subject invention, in a specific embodiment, to be manufactured for single patient use thereby eliminating the requirement for cleaning, special care, the maintenance of expensive inventory, and most importantly eliminating the opportunity for cross contamination between patients. A further aspect of the subject invention concerns a novel sheath which can cover all or a portion of the parts of the imaging system which enter the patient. This sheath can reduce the need for expensive sterilization of the subject intubation scope after use.

An embodiment of the subject imaging system utilizing plastic optical fiber is highly advantageous because of its longer life, increased ruggedness, greater flexibility, comparable image quality, optional disposability, and lower cost, compared to glass scopes. These scopes are useful, for example, for observing the bronchi of the lungs, locating the tracheal opening to allow insertion of an endotracheal tube into the trachea for intubation, and visually locating the endotracheal tube tip. Specifically, using a scope of the subject invention, a practitioner can easily and precisely identify the exact location of the distal end of the tracheal tube, as well as the various anatomical airway landmarks. Currently, this degree of precision is only possible with an expensive glass fiberoptic bronchoscope. In addition, the scopes of this invention are particularly advantageous for use in anesthesiology.

In a specific embodiment, the subject imaging system can incorporate a malleable stylet which can retain its shape when bent. This embodiment can be used as an intubation scope. During an intubation, an anesthesiologist can insert the subject intubation scope into an endotracheal tube to be inserted into a patient. Once the intubation scope is inserted into the endotracheal tube, the anesthesiologist can bend the intubation scope - endotracheal tube combination into a shape, essentially any shape, which facilitates insertion into the patient. Advantageously, the anesthesiologist can then hold the scope-tube combination in one hand, with a conventional grip, for example in a pen-like fashion, and easily maneuver the scope-tube combination during insertion into the patient. This is facilitated as a result of the center of balance of the scope/stylet-tube combination being in approximately the center ⅓ of the length of the scope/stylet-tube combination.

While prior art scopes have required the anesthesiologist to change the standard procedure for inserting an endotracheal tube, for example by holding on to the scope and/or manipulating a handle on the scope to direct the tip of the scope, an anesthesiologist using the subject scope can hold the endotracheal tube in the conventional manner during intubation. This is an important improvement over the prior art because anaesthesiologists are not necessarily trained in endoscopy and without such additional training, would not normally be adept with the techniques for use of such prior scopes. The subject technology can therefore be referred to as a transparent technology, due to the minimal instruction needed before use thereof. Specifically, the subject invention allows an anaesthesiologist to hold the scope-tube combination with one hand during intubation and, for example of the lenght le, use a laryngoscope with the other hand, if necessary. In addition, the subject intubation scopes can be removed from the endotracheal tube, after insertion, with only one hand, thus freeing the anaesthesiologist's other hand. This is in contrast to many prior art scopes which require two hands for use, for example due to a twist motion needed to unlock the scope from the endotracheal tube.

In a specific embodiment, the subject invention pertains to a plastic optical fiber imaging scope, having an optional sheath, for intubation. This intubation scope can be used for intubation of patients under general and/or local anesthesia. The intubation scope of this embodiment can be disposable, sterilizable for reuse, or enclosed within a disposable sheath for reuse without expensive sterilization. Due to lower cost for the plastic optical fiber scopes, the scopes of the subject invention are particularly advantageous for situations calling for disposable scopes, and can be less expensive than the cleaning and sterilization costs for existing glass scopes.

Imaging scopes with varying components and corresponding performance capabilities can be manufactured with this new technology. By way of these multiple embodiments, the subject invention can be used for imaging essentially all of the airway system of humans and animals.

### BRIEF DESCRIPTION OF THE DRAWINGS

FIG. 1 shows an embodiment of the intubation scope of the subject invention.

FIGS. 2A and 2D illustrate a longitudinal cross section and a transverse cross section, respectively, of the distal end of a sheath designed to fit over a plastic optical fiber image guide.

FIGS. 2B and 2E illustrate a longitudinal cross section and a transverse cross section, respectively, of the distal end of a sheath designed to fit over a plastic optical fiber image guide, wherein the sheath comprises an illumination fiber.

FIGS. 2C and 2F illustrate a longitudinal cross section and a transverse cross section, respectively, of the distal end of a sheath designed to fit over a stylet incorporated with a plastic optical fiber image guide, wherein the sheath comprises an illumination fiber.

FIG. 3 illustrates markings and a hollow channel for ventilation, suction, and/or irrigation, in accordance with the subject invention.

FIGS. 4A and 4D illustrate a longitudinal cross section and a transverse cross section, respectively, of the distal end of an embodiment of the subject invention comprising a stylet, an image guide, at least one illumination fiber, and rings to hold the components together.

FIGS. 4B and 4E illustrate a longitudinal cross section and a transverse cross section, respectively, of the distal end of an embodiment of the subject invention comprising a stylet, an image guide, at least one illumination fiber, and an outer covering to hold the components together.

FIGS. 4C and 4F illustrate a longitudinal cross section and a transverse cross section, respectively, of the distal end of a flexible embodiment of the subject invention comprising an image guide, at least one illumination fiber, and an outer covering to hold the components together.

FIG. 5 illustrates a specific embodiment of the subject invention comprising an image guide, at least one illumination fiber, and a stylet made of multiple wire strands.

FIG. 6 illustrates an embodiment of the subject invention wherein an image guide, an illumination fiber(s), and a hollow channel are housed within a malleable stylet.

FIG. 7 illustrates a preferred embodiment of the subject invention incorporating an image guide, an illumination fiber(s), and a malleable stylet, held together by an outer covering.

FIG. 8 is a transverse cross-sectional view of a preferred embodiment of a malleable stylet/scope combination according to the subject invention with U-channel into which a scope snaps. Scope in this representation has an imaging guide, two illumination fibers and one suction/insufflation/irrigation lumen. Drawing is approximately to scale and the standard diameter stylet is moved off-center with respect to the longitudinal axis within its sheath to accommodate the U-channel. Note that the imaging guide lumen in the scope is shown as the lumen furthest away from the center of the stylet and closest to the internal wall of the endotracheal tube (ETT), which may promote an undesirable fish-eye view. Ideally, the scope would be indexed such that the imaging guide is as close as possible to the center of the ETT when the scope/stylet combination is loaded into the ETT.

FIG. 9 is a transverse cross-sectional view of yet another preferred embodiment of a stylet/scope combination according to the subject invention with C-channel into which a scope snaps. Scope in this representation has an imaging guide, two illumination fibers and one suction/insufflation/irrigation lumen. Drawing is approximately to scale. The stylet transverse cross-section is made elliptical to accommodate the deeper C-channel and is moved off-center with respect to the longitudinal axis within its sheath to accommodate the C-channel. C-channels where the two edges of the channel almost meet are also contemplated, are within the scope of the subject invention, and are readily envisioned by the ordinary artisan in view of the teachings herein.

FIG. 10 is a transverse cross-sectional view of yet another embodiment of a stylet/scope combination according to the subject invention with C-channel into which a scope snaps. Scope in this representation has an imaging guide, two illumination fibers and one suction/insufflation/irrigation lumen. Drawing is approximately to scale. The stylet transverse cross-section is made crescent-shaped to accommodate a deeper C-channel and is moved off-center with respect to the longitudinal axis within its sheath to accommodate the C-channel. C-channels where the two edges of the channel almost meet are also contemplated, are within the scope of the subject invention, and are readily envisioned by the ordinary artisan in view of the teachings herein.

FIG. 11 is a transverse cross-sectional view of still another embodiment of a stylet/scope combination according to the subject invention with C-channel into which a stylet snaps. Scope in this representation has an imaging guide and two illumination fibers. Drawing is approximately to scale. The stylet transverse cross-section and diameter as depicted, although not necessarily required by this embodiment, are those of a standard stylet.

FIG. 12 is a transverse cross-sectional view of yet another embodiment of a stylet/scope embodiment according to the subject invention with a slot-channel into which a scope snaps. Scope in this representation has an imaging guide and an illumination fiber. Drawing is approximately to scale. The shape of the scope provides indexing of the scope relative to the stylet.

FIG. 13 is a transverse cross-sectional view of still another embodiment of a stylet/scope combination according to the subject invention that provides positive indexing of the scope relative to the stylet.

FIG. 14 is a transverse cross-sectional view of a still further embodiment of a scope/tube/stylet combination according to the subject invention wherein there is a scope receiving channel on the external surface of the airway device running substantially the length of the device.

FIG. 15 is a transverse cross-sectional view of yet a further embodiment of a scope/tube/ stylet combination according to the subject invention wherein the airway device comprises a channel on the internal surface of the airway device running substantially the length of the device.

### DETAILED DISCLOSURE OF THE INVENTION

The subject invention utilizes optical fiber to produce fiber optic scopes, and in a particular embodiment, intubation scopes, for use in imaging the human airway. In a preferred embodiment, the subject invention utilizes plastic optical fiber. The device of the subject invention can assist practitioners in properly introducing and confirming the position of tracheal tubes. Although the device has application in “difficult” intubations, use in all intubations is envisioned for confirming optimal tracheal tube advancement between the vocal cords and positioning within the trachea. This can only be achieved with direct tracheal visualization. The plastic fiberoptic device of the subject invention makes this possible while also being highly cost effective.

The device of the subject invention has application for intubating patients undergoing general, intravenous and local anesthesia and in emergency situations. Thus, the device can be used in surgical procedures as well as in intensive care units, emergency departments and the prehospital settings.

The device of the subject invention can incorporate plastic fiberoptic technologies, enabling direct visualization of the pharynx, glottic opening, larynx and trachea and thus, facilitate accurate tracheal tube placement and periodic verification of tracheal tube tip location. Further, the subject device can easily be steered, simplifying proper tube placement. Traditional reusable glass fiber bronchoscopes are expensive to purchase and maintain. Effectively cleaning the bronchoscope is difficult. It is also recognized that sterility of existing reusable glass fiber bronchoscopes is often not achieved after use with a patient.

The subject invention achieves substantial improvements in performance compared to existing glass fiber scopes, including: (1) longer life; (2) increased ruggedness; (3) greater flexibility; (4) optional disposability; (5) greater ease of use; and (6) less expense. By varying the components of the intubation scopes utilizing the teachings of the subject invention, these performance characteristics can be optimized, to facilitate the use of these scopes for imaging a vast portion of the human airways and in a variety of situations. Thus, the scopes of the subject invention can be used, for example, for observing the bronchi of the lungs, locating the tracheal opening to allow insertion of an endotracheal tube into the trachea for intubation, and locating the endotracheal tube tip. In particular, an anesthesiologist can, using the scope of the subject invention, stand behind the head of a patient while performing an intubation. Additionally, this device will reduce the force necessary for laryngoscopy. Reduced force leads to less tissue trauma, hemodynamic changes, and post-operative sore throat complications. Advantageously, the scopes of the subject invention are more cost effective in situations requiring a disposable scope and/or can be used with an optional disposable sheath to enclose the portion of the scope entering the body to reduce cleaning and sterilization costs.

In one embodiment the light source can be derived from a standard laryngoscope. In a second embodiment, one or more optical fiber(s) can transmit the light required to illuminate the airway. The source of the light can be, for example, a laryngoscope or an inexpensive separate light source.

Image guides used in conjunction with intubation, for example, bronchoscopes, are typically made with step index glass optical fiber. Plastic optical fiber can also be fabricated with a step index of refraction. Both plastic and glass step index fibers are constructed with a core of refractive index n₁, and a cladding of refractive index n₂, where n₁>n₂. A second type of fiber is known as gradient index or graded index fiber and can also be made with plastic or glass. Since flexibility is an important characteristic for the scopes of the subject invention, plastic gradient index optical fibers are preferred over glass gradient index optical fibers for the subject image guides.

In comparing the step index structure with the gradient index structure, it is noted that there are different trajectories of light rays in these two fiber structures. Within step index fiber, the light travels in straight lines, and is reflected at the core-cladding interface. While in gradient index fiber, the light travels in a curved trajectory always being refracted back towards the axis of the fiber. As a consequence, an image can be conveyed within a single gradient index fiber, while an image cannot be conveyed within a single step index fiber. Although, when conveying an image in a single gradient index fiber, some correction maybe required to correct aberrations in the output image, for example, by using one or more negative gradient index lens attached to one or both ends of the single gradient index fiber.

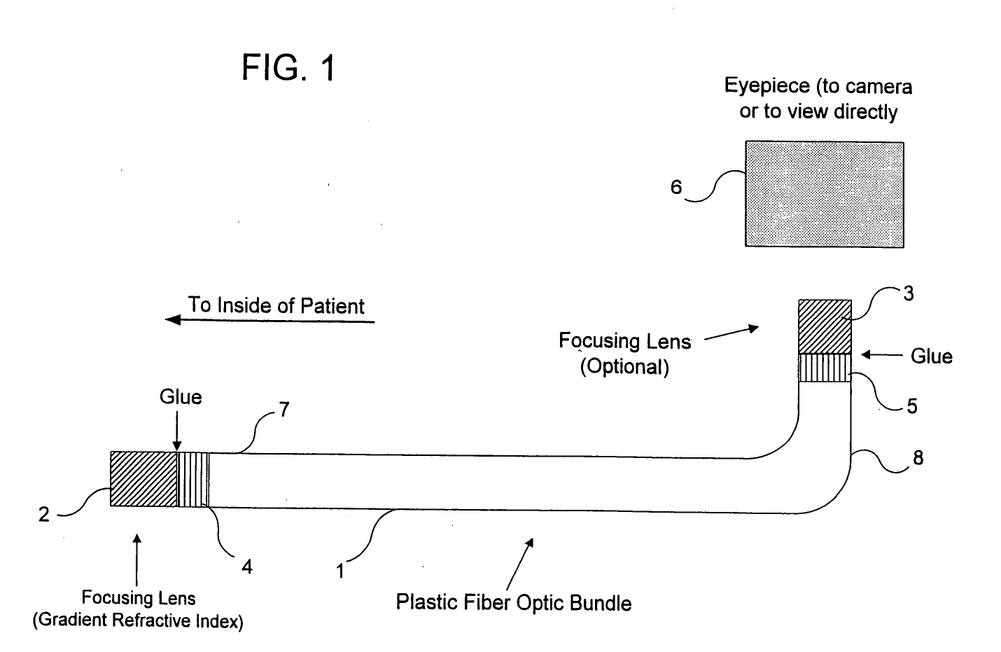

Referring to FIG. 1, the intubation scope of the subject invention comprises an image guide 1, which conveys optical images from inside the body, for example from inside the human airways, to outside of the body for viewing by a medical caregiver. In a specific embodiment, this image guide 1 can comprise a single gradient index plastic optical fiber. In a specific embodiment, this single fiber can have a diameter, for example, of about 0.5 to 2.0 mm. A focusing lens 2 can optionally be used to focus the desired image into the distal tip 7 of the single fiber, the distal tip 7 entering the body, generally through the mouth or nose. This lens 2 can be attached by, for example, optical glue, and can act as a bi-convex lens to focus the desired image onto the distal tip of the single fiber. In this single fiber embodiment, a negative gradient rod lens, made of glass or plastic, can be attached to the proximal end of the single fiber image guide 1 and used to correct for aberrations resulting from the use of a single gradient index plastic optical fiber to carry the image.

In an alternative embodiment, this image guide 1 can comprise a bundle of plastic optical fibers. This plastic fiber optic image guide 1 can be made of a plurality of individual plastic optical fibers which have been fused together. In a specific embodiment, this bundle can comprise approximately 10,000 individual plastic optical fibers, wherein the bundle is approximately 1.0 millimeter in diameter. This bundle can comprise gradient index plastic optical fibers and, in a preferred embodiment, this bundle can comprise step-index plastic optical fibers. Each end of the bundle can be polished to allow high resolution imaging. A focusing lens 2 can be used to focus the desired image onto the distal tip 7 of the bundle, the distal tip 7 entering the human body, generally through the mouth or nose. This lens 2 can be attached at the distal tip 7 of the bundle, for example, by optical glue 4. This lens 2 can act like a bi-convex lens to focus the image onto the distal tip of the fiber optic bundle. In a preferred embodiment, the lens 2 can be a gradient-index glass rod lens. In a more preferred embodiment, the lens 2 can be a gradient-index plastic rod lens.

An optical system can transmit the image from the proximal end of the fiberoptic image guide 1 onto a charge coupled device (CCD). The CCD can be used to convert the image into an electrical signal which can be displayed on a monitor. Alternatively, camera equipment available to the physician can be used. In a specific embodiment, the proximal tip 8 of the image guide 1 is arranged so that the image from inside the patient is focused directly onto an imaging eyepiece 6. The imaging eyepiece 6 can be connected to a camera for viewing the image, or the image can be viewed directly by the medical caregiver through the imaging eyepiece 6.

In an alternative embodiment, when a camera is not to be used for viewing the image, a second lens 3 can be attached, for example, by optical glue 5, to the proximal tip 8 of the image guide 1, the proximal tip remaining outside of the body. This lens can be, for example, a mini plastic lens microscope connected directly to the proximal end of the fiber scope for direct viewing by the caregiver, i.e., by placing an eye to the microscope lens. In a specific embodiment, the fiberoptic image guide can be lengthened and mini plastic lens microscope can be mounted on a caregiver's head, for example, by a mounting means such as a pair of glasses or goggles. This allows the caregiver to have both hands free to perform, for example, an intubation, while being able to view the patient and the image from the scope without having to turn his or her head.

Manufacturing the subject invention without a camera can be done to reduce costs or to meet the needs of certain medical situations. However, when the eyepiece is connected to a camera, the image can be displayed via, for example, a television monitor. This monitor can be, for example, placed out of the sterile field for viewing.

In a specific embodiment, the visualization system scope of the subject invention can be inserted into a plastic tube (sheath), which can have a transparent end plate. This combination can then be used for imaging the airway. The image viewed through the end plate is unimpaired by the sheath or end plate. The advantage of this sheath is that it is disposable and allows the intubation scope to be reused with minimal sterilization.

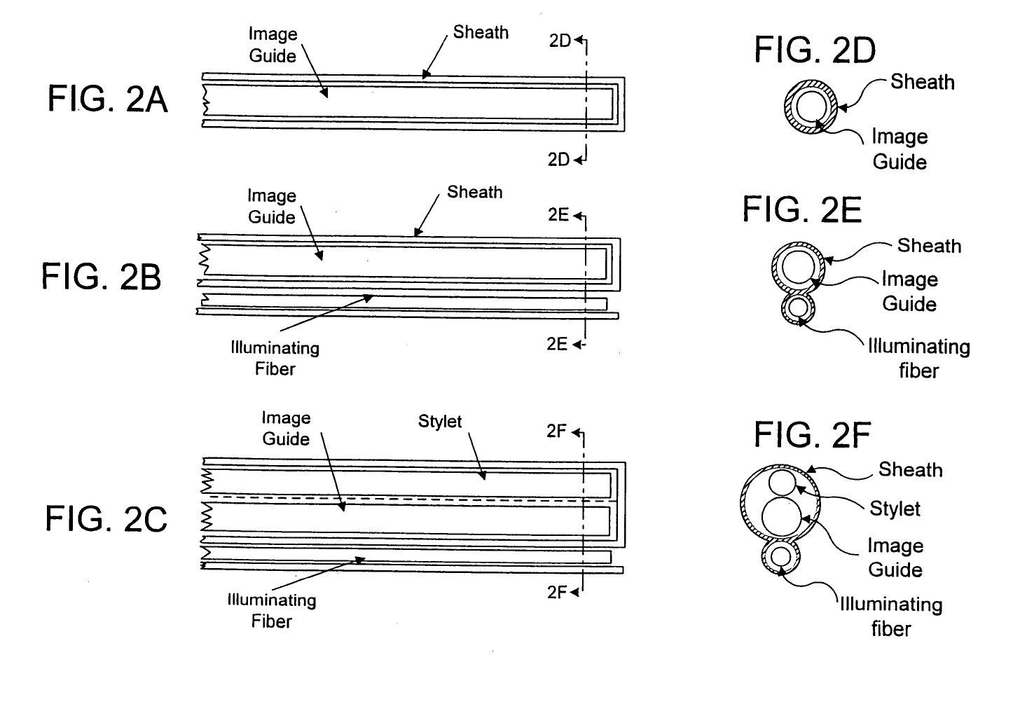

In a preferred embodiment, the sheath can have at least one internal, or external, illuminating optical fiber(s) which transmits light to illuminate the internal body structure to be imaged. Additionally, it is preferred but not essential, that there be no transparent end plate at the distal end of the illuminating optical fiber(s) to avoid the illuminating light reflecting at such a plate and impairing the quality of the image. A longitudinal cross section and a transverse cross section of a sheath comprising an external illumination fiber are shown in FIGS. 2B and 2E, respectively.

When performing, for example, an intubation, a malleable stylet, typically made of metal, is often used in conjunction with an endotracheal tube to facilitate the placement of the tube into the body. In a specific embodiment, the subject intubation scope can incorporate a stylet, wherein the stylet can be bent into the shape which the caregiver believes will facilitate the easiest and safest placement of the endotracheal tube, and then the endotracheal tube, which typically surrounds the stylet and scope, can be inserted into the patient. A longitudinal cross section and a transverse cross section of a sheath comprising an external illumination fiber, where the sheath is designed to fit over a stylet incorporated with a plastic optical fiber image guide, are shown in FIGS. 2C and 2F, respectively. In this case, the sheath and illuminating fiber could be regarded as disposable after a single use.

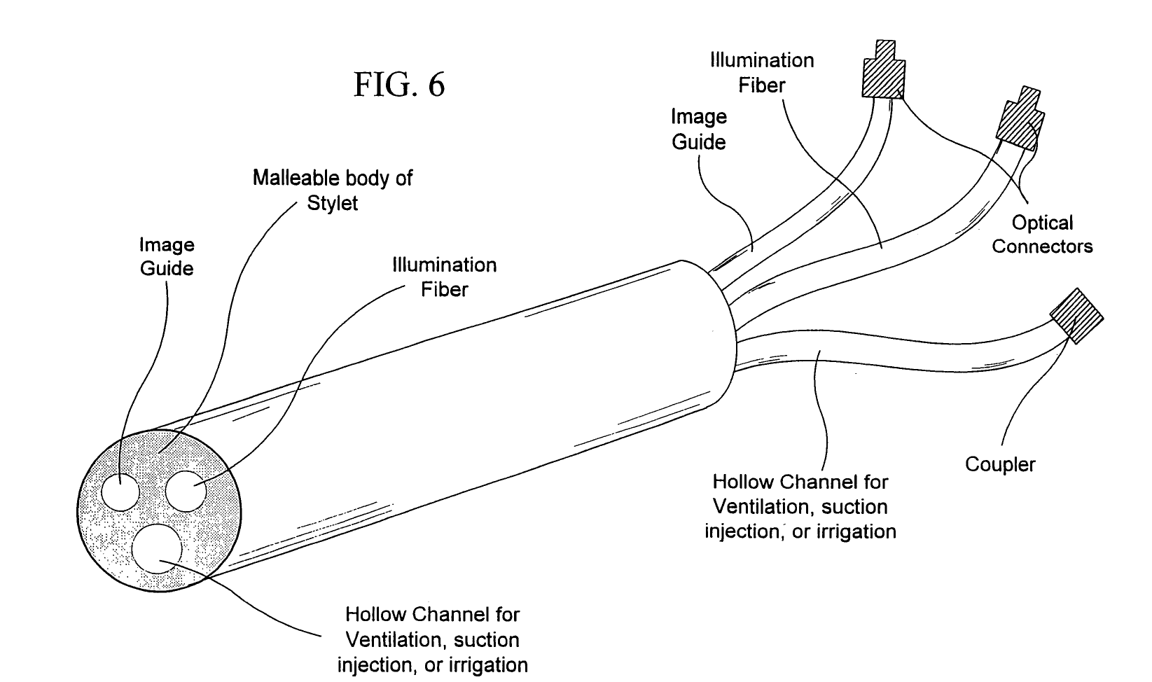

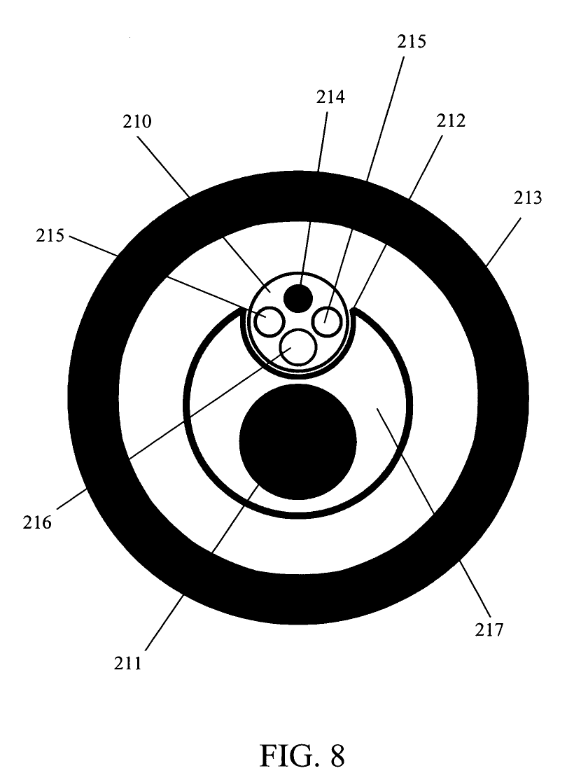

Accordingly, the intubation scope and/or sheath of the subject invention can comprise such a stylet, such that many combinations of scope, stylet, illuminating fiber(s), and sheath are possible. In accordance with the subject invention, the combination of an image guide and a malleable stylet, which retains its shape when bent, facilitates the placement of an endotracheal tube into a patient. In a preferred embodiment, a solid metal stylet, for example a conventional endotracheal tube stylet, can be utilized. The image guide can be attached to the stylet such that the image guide takes essentially the same shape as the stylet when the stylet is bent. In a specific embodiment, referring to FIG. 6, the image guide can be incorporated into the stylet. Other embodiments, such as, for example, those depicted in FIGS. 8-13, the image guide can be disengagably coupled to the stylet. Accordingly, the stylet-image guide combination, i.e., the intubation scope, can be inserted into an endotracheal tube which is to be inserted into a patient. In a more preferable embodiment, the intubation scope can also comprise an illumination fiber, for example, also attached to the stylet. Once the intubation scope is inserted into the endotracheal tube, a caregiver can bend the endotracheal tube into the shape, essentially any shape, which the caregiver believes will facilitate the easiest and safest insertion of the endotracheal tube. In fact, a caregiver can insert the tube-scope combination into a patient using one hand to hold and maneuver the tube-scope combination. Once the tube-scope combination is in place in the patient, a caregiver can, again with one hand, remove the subject intubation scope from the endotracheal tube.

A variety of attachment means can be utilized in accordance with the subject invention to attach the stylet, image guide, and/or illuminating fiber. It is preferred that the relative position of the distal tip of the image guide and the distal tip of the illumination fiber remain fixed, both in angular orientation and axial translation, in order to enhance the quality of the image. In addition, it is preferred that the relative position of the distal end of the stylet also remain fixed with respect to the distal tip of the image guide.

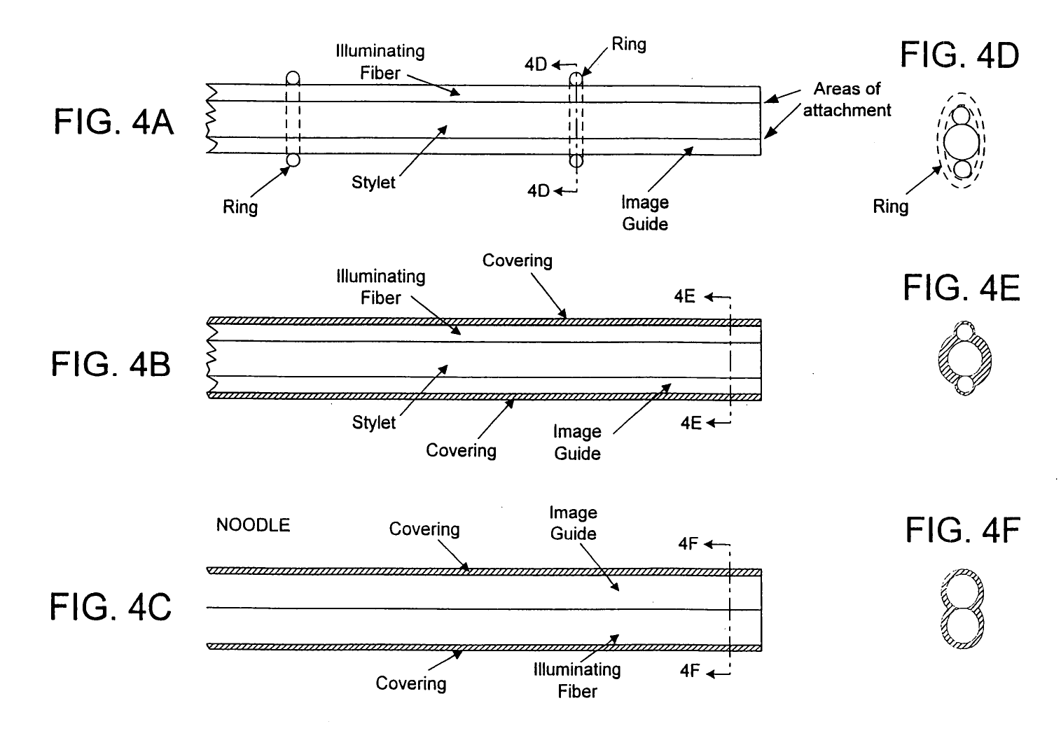

Referring to FIGS. 4A and 4D, a specific embodiment of the subject invention is shown where the distal ends of a stylet, an image guide, and an illumination fiber are securely attached to each other via an attachment means, for example with glue or a clip. This embodiment also includes rings, for example made of an appropriate elastic material, which hold the various components together along the length of the intubation scope. These rings can allow for slippage of the various components, for example during the bending of the intubation scope, while maintaining the relative positions of the distal ends of the various components.

Alternatively, referring to FIGS. 4B and 4E, an outer covering can be used to hold the various components together. This outer covering can be made of a material, for example a polymer, which can alter, either increase or reduce, the friction between the subject intubation scope and the inside of an endotracheal tube. For example, reduced friction can facilitate the removal of the subject intubation scope after intubation of a patient. Again, it is preferred that the relative positions of the distal ends of the various components remain constant. In an additional embodiment, a transparent end covering can be utilized to cover the distal tip of the intubation scope.

FIGS. 4C and 4F show an embodiment of the subject invention which can be referred to as a noodle. This embodiment does not incorporate a stylet and, therefore, can be useful, for example, for nasal intubations. This embodiment can also be used, for example, to check the position of the distal tip of an endotracheal tube which is in place in a patient.

Stylets of various cross-sectional shapes can be used in accordance with the subject invention. A variety of materials for producing the stylet are also envisioned within the scope of the subject invention, for example polymers, metals, or other materials having the proper physical characteristics. It is important for the stylet to be malleable such that the stylet can be bent and will retain its shape when bent. Preferably, the subject stylet can be bent easily by a user into essentially any shape which will facilitate the insertion.

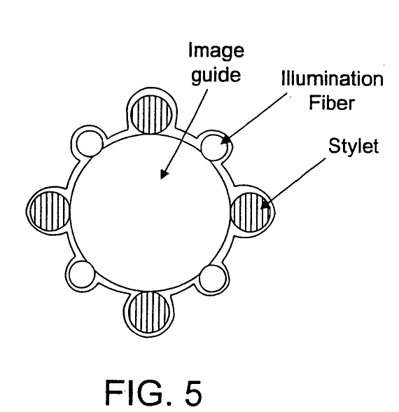

In addition, the subject intubation scopes can have a coating, for example a polymer coating. In a specific embodiment, this coating can be placed around a solid cylindrical rod made of a malleable metal. The coating can be used to modify friction between the endotracheal tube and the subject scope so as to optimize movement of the scope within the tube. For example, a low friction coating may decrease friction between the scope and the endotracheal tube so as to ease withdrawal of the scope from the tube. Various cross-sectional shapes for the subject intubation scopes are also possible. For example, referring to FIG. 5, the subject scope can incorporate a stylet comprising multiple, thinner rods which can be positioned circumferentially around the image guide. If illumination fibers are utilized, these illumination fibers can also be positioned circumferentially around the image guide, for example interspaced with the multiple rods of the stylet. In addition, a coating and/or an outer covering can be applied to the outside of the intubation scope to assist in holding the multiple stylets and multiple illumination fibers to the side of the image guide. This coating may allow for reuse of the subject scope, for example after cleaning.

In an additional embodiment, referring to FIG. 6, an outer covering around the subject image guide can act as a stylet, for example if the outer coating is malleable and retains its shape when bent. The embodiment shown in FIG. 6 comprises a malleable stylet having hollow channels where an image guide and illumination fiber(s) can reside. This stylet can, for example, be extruded such as to have these channels. As also shown in FIG. 6, an additional channel can be provided for injection, ventilation, suction, and/or irrigation. In a preferred embodiment, the subject imaging scopes have no moving parts other than those resulting from bending of the stylet, image guide, any illumination fibers, and other lumens.

Preferably, during insertion of an endotracheal tube into a patient, the distal tip of the image guide can reside near the distal tip of the endotracheal tube such that a caregiver receives images from near the distal tip of the endotracheal tube, so as to facilitate proper placement of the tube. In addition, it is preferable that the distal tip of the stylet and image guide do not extend from the distal tip of the tube where they may injure the trachea or other areas during the insertion of the tube. To prevent the distal tips of the stylet and image guide from traveling past the distal tip of the tube, the stylet can be bent near the proximal end of the tube, for example bent over the proximal end of the tube. The image guide can be bent as well, for example if attached to the stylet of the scope at the proximal end of the tube, or can move independently from the stylet.

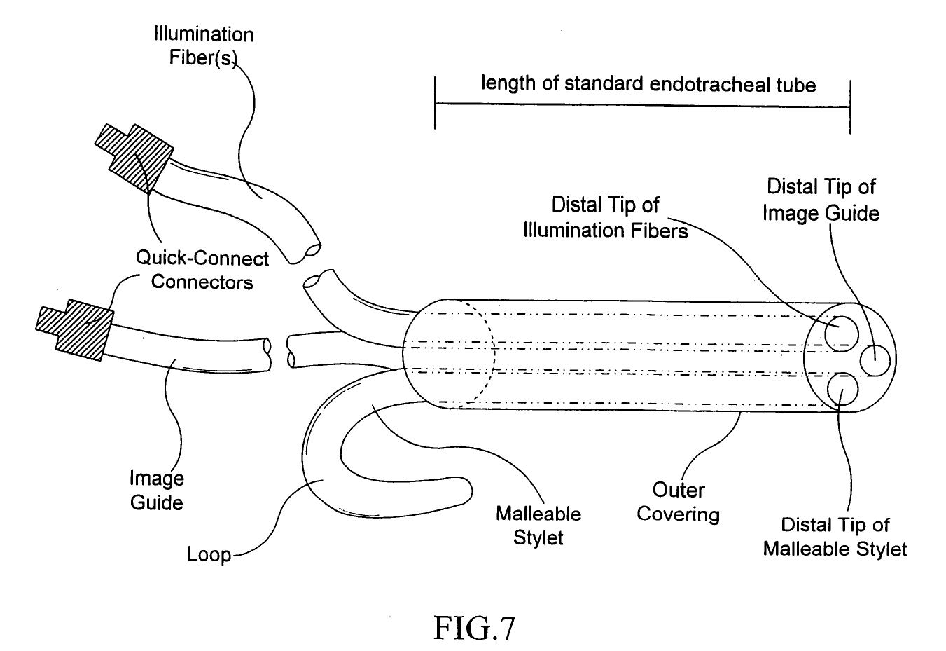

In a preferred embodiment of an intubation scope in accordance with the subject invention, as illustrated in FIG. 7, the proximal end of the stylet can terminate, for example in a loop, at a length slightly longer than an endotracheal tube into which it is to be inserted. Such a loop can allow a caregiver to easily grasp the stylet end to bend the stylet end over the proximal end of the tube. In addition, a looped proximal end of the stylet can assist in removal of the scope from the tube. For example, a user can hold the end of the tube with one hand and use the thumb on the same hand to move the scope out of the tube by hooking the loop with the thumb. Advantageously, due to the lightweight image guide and illumination fiber(s), the center of gravity of the endotracheal tube - intubation scope combination can be located in essentially the same location along the endotracheal tube as with a conventional stylet inserted in an endotracheal tube; that is, preferably, approximately the center ⅓ portion if the tube is envisioned as having a proximal ⅓, a distal ⅓, and a center ⅓; such as to provide good balance in the user's hand during intubation. This allows the center of balance of the tube-scope/stylet combination in a user's hand to remain in essentially the same position as for a conventional endotracheal tube-stylet combination.

In accordance with the subject invention, the ability of a caregiver to hold the endotracheal tube itself during insertion into a patient has many advantages. By holding the tube with a conventional, or pen-like, grip a caregiver can twist or rotate the tube, as well as move it translationally, with more leverage than if held by, for example, a scope inserted into the tube. During the insertion, a caregiver can view the image from the image guide on, for example, a monitor, without the need to view the patient directly.

In addition, the ability to bend the scope-tube combination in essentially any shape prior to insertion of the tube into a patient can reduce the risk of injury to a patient whose anatomy may require a tailored bend. Advantageously, insertion of the tube can then be performed on a patient in the neutral position. Prior art devices have required the patient to be in a “sniffing” position or to have the head tilted back to create a straighter insertion channel, due in part to the stiffness and limited range of motion of the prior devices. Insertion of the tube while a patient is in the neutral position, as opposed to the sniffing or head-tilted-back positions, results in reduced increases in blood pressure, heart rate, and reduced injury to the treachea. In addition, the ability to bend the scope-tube combination in essentially any shape allows the tube to be preformed to a shape tailored specifically to a patient, prior to insertion of the tube. This allows insertion of the scope-tube combination while a patient is in the neutral position, and can thus eliminate the need for a laryngoscope to elevate tissues. If preferred, a laryngoscope can be used to move the tongue out of the way such that the tube-scope combination can be inserted. However, there is no need to use the laryngoscope to further elevate tissues, for example, to see the larynx. The subject tube-scope is preferably rigid enough so that a laryngoscope is not necessary. The stylet/scope-tube can sweep the tongue away, eliminating the need for a laryngoscope, thus reducing complications, such as chipped teeth. In addition, less force can be applied, reducing injuries. This allows intubation to be performed while a patient is awake or asleep, in an elective or emergency situation. In addition, the ability to intubate a patient in the neutral position can prevent further injury to a patient with, for example, known or possible neck or spine injuries. Patients with known or possible cervical spine instability or fused/fixed spine can be intubated and/or examined without having to be moved.

In a specific embodiment in accordance with the subject invention, an intubation scope can have a removable stylet. The stylet can be removed to perform, for example, nasal intubation or stoma/tracheotomy. In addition, this embodiment can allow for insertion of an endotracheal tube, utilizing the intubation scope with the stylet attached, and the subsequent verification of the tube's position, utilizing the intubation scope with the stylet removed. In another embodiment, the subject scope can have no stylet. This embodiment can be useful for confirmation of the endotracheal tube location and inspection of patients' tissues and structures, for example to reduce the need for x-rays. FIGS. 4C and 4F illustrate one such embodiment.

In a specific embodiment of the subject intubation scope, a charge-coupled device (CCD) can be mounted at the distal tip of the stylet, which can eliminate the necessity for an optical fiber image guide. The signal generated from the CCD can be carried, for example via wires, to a camera external to the endotracheal tube. This camera can be located in any position which allows a caregiver to conveniently view the camera during intubation of the patient. In a further embodiment, the CCD and camera can be located at the proximal end of the stylet, such that an image is carried from the distal tip of the endotracheal tube to the CCD by an image guide during intubation.

Once the subject intubation scope is inserted into an endotracheal tube and secured in place, for example by bending the proximal end of the stylet over the proximal end of the endotracheal tube, the image carried from the distal tip of the image guide to the proximal end of the image guide needs to be viewed by the caregiver. In a preferred embodiment, the proximal end of the image guide can terminate with a quick-connect connector such that the proximal end of the image guide can easily plug into a device for receiving and displaying the image. Quick-connect connectors are particularly advantageous when a plastic optical fiber image guide and plastic optical fiber illumination fibers are utilized, due to greater misalignment tolerances than with glass optical fiber. The proximal end of any illumination fibers can also terminate with quick-connect connectors such that easy connection to an illuminating light source is possible. In a specific embodiment, slide and lock quick-connect connectors may be utilized. The use of quick-connect connectors allows for a free flexible tail and a lightweight intubation scope. The use of slide and lock quick-connect connectors can assist a caregiver in maintaining proper orientation of the image being viewed and prevent the connector from coming loose during operation, thus enhancing performance.

A preferred embodiment of an intubation scope in accordance with the subject invention is illustrated in FIG. 7. The proximal ends of the illumination fiber and image guide can form a free flexible tail which is lightweight. Accordingly, after insertion of the subject intubation scope into an endotracheal tube, the scope-tube combination can be maneuvered easily and has the same feel in a user's hand as the conventional tube with a conventional stylet inserted. The quick-connect connectors can be connected just before placement of the scope-tube combination into the patient, allowing the caregiver considerable freedom of maneuverability while inserting the intubation scope into the endotracheal tube.

The intubation scope and method of tracheal tube placement verification of the subject invention represents a significant cost saving for the hospital. Rather than having to periodically x-ray the patient to verify tube position, the hospital staff can use the subject invention to not only confirm proper tube placement but to also evaluate the airway for obstruction and/or erosion, which is not possible with x-rays. Advantageously, the use of the subject technology can reduce a patients exposure to x-rays.

The device of the subject invention is easily connected to bronchoscope imaging equipment, without incurring the cost of buying and sterilization processing of the bronchoscope. In another embodiment, an off the shelf LCD device similar to a Sony “WATCHMAN” can be utilized for imaging.

Following are examples which illustrate procedures for practicing the invention. These examples should not be construed as limiting.

### EXAMPLE 1

The device of the subject invention not only makes intubation easier and more accurate, but also reduces the potential for inflicting injury with the laryngoscope blade. Using the device of the subject invention, the practitioner only needs to use the laryngoscope blade to control the position of the patient's tongue. The device can also be used without a laryngoscope and for nasotracheal intubation.

The device of the subject invention can comprise a bundle of 10,000 individual plastic optical fibers (bundle is approximately 1.0 millimeter in diameter). The resolution of the image is comparable to an existing glass fiberoptic bronchoscope, providing good visualization of the patient's airway.

The manufacturing process places the bundle integral to a standard malleable stylet. An additional fiber may be used for illuminating purposes. Suctioning and insufflation can be added by adding an additional channel if desired.

A focusing lens (i.e., gradient refractive index lens) is placed at the distal tip of the optical fibers and acts like a bi-convex lens to focus the image onto the distal tip of the fiber optic bundle. An optical system transmits the image from the proximal end of the fiberoptic bundle onto a charge coupled device (CCD). The CCD is used to convert the image into an electrical signal which can be displayed on a monitor. Alternatively, camera equipment available to the physician may be used.

### EXAMPLE 2

Referring to FIGS. 2A, 2B, 2C, 2D, 2E, and 2F, this example provides three illustrative combinations of image guide, illuminating fiber, stylet, and/or sheath. FIGS. 2A and 2D illustrate a longitudinal cross section and a transverse cross section, respectively, of the distal end of a sheath designed to fit over a plastic optical fiber image guide. The sheath covers the distal tip of the image guide with a transparent end plate. In this case, any illuminating fibers and/or stylets would not be enclosed within this sheath, although they could have their own sheaths.

FIG. 2B and 2E illustrate a longitudinal cross section and a transverse cross section, respectively, of the distal end of a sheath designed to fit over a plastic optical fiber image guide, wherein the sheath comprises an illumination fiber. The distal end of the illumination fiber is not covered by the sheath, in this example, so as to not impair the image. Accordingly, the illuminating fiber can be disposed of with the sheath. In this embodiment, the sheath acts to attach and position the illumination fiber with respect to the image guide.

FIGS. 2C and 2F illustrate a longitudinal cross section and a transverse cross section, respectively, of the distal end of a sheath designed to fit over a stylet incorporated with a plastic optical fiber image guide, wherein the sheath comprises an illumination fiber. The distal end of the illumination fiber is not covered by the sheath, but the distal end of the image guide plus stylet is covered. In this embodiment, the stylet can be reused along with the image guide. Other geometrical arrangements of the stylet, image guide, and illumination fiber are obviously possible.

### EXAMPLE 3

It is sometimes necessary to replace an endotracheal tube in a patient, for example when the tube no longer provides an adequate airway. In a specific embodiment, the subject invention can permit the easy removal of one endotracheal tube and its replacement with a new one.

A device in accordance with the subject invention can be inserted into the patient's trachea, generally through an existing tracheal tube, until the distal tip is at or near the tip of the tube. Oxygen for ventilation, suction or irrigation can also be applied in succession via, for example, quick fit connectors to the proximal end if desired. Once the anatomy is visualized, the light source, camera or eyepiece, and oxygen/suction/irrigation can all be disconnected from the proximal end of the subject device. The endotracheal tube can then be completely removed by sliding it out of the trachea, along, and off the proximal end of the subject device. The connectors of the subject device can be of a suitable diameter and located at different positions relative to the proximal end to permit their passage through the tracheal tube. The new tracheal tube can be loaded onto the subject device and the illumination and visualization equipment reconnected. The tracheal tube can be advanced down into the trachea while observing the airway anatomy via the subject device. Preferably, the subject device can be rigid enough to maintain its location in the trachea as it guides a replacement endotracheal tube into the trachea.

Alternatively, the image guide and illumination fiber(s) can remain connected until the endotracheal tube to be replaced has been slid out of the trachea. This allows continuous monitoring of the location of the distal tip of the tube exchanger, reducing the risk of the tube exchanger being dislodged during removal of the endotracheal tube. Once the tube is completely out of the patient, the proximal ends of the image guide and illumination fiber(s) can be disconnected to slide the tube off of the exchanger and slide a new tube onto the exchanger. The image guide and illumination fiber can then be reconnected to confirm the position of the exchanger before and during the placement of the new tube into the patient.

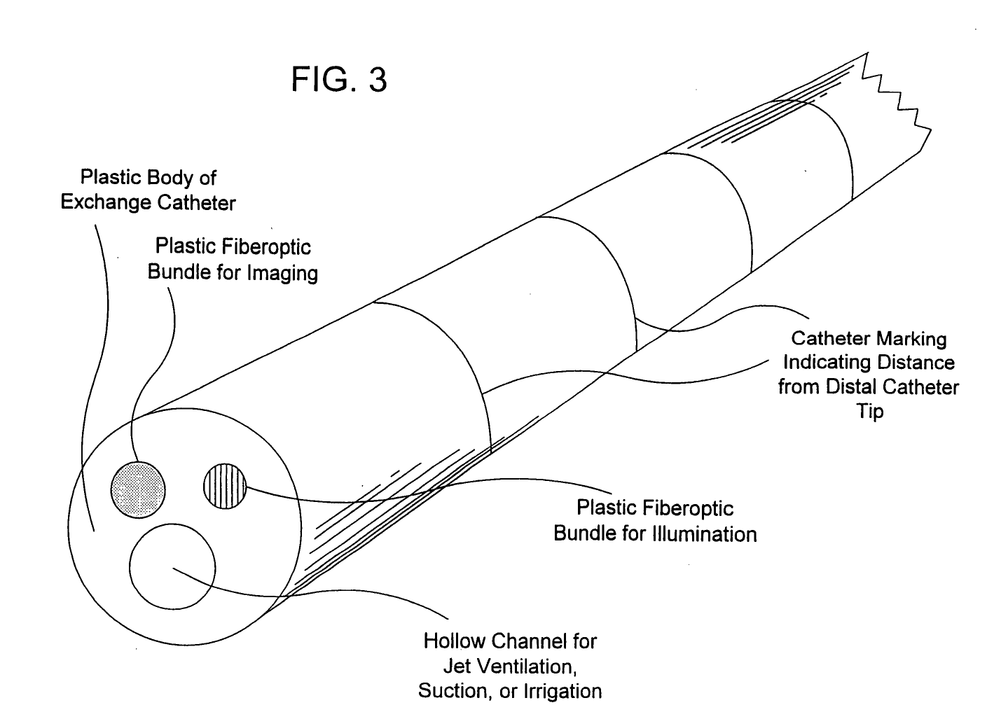

In order to utilize the subject invention during the exchange of an endotracheal tube, the fiber optic bundle can, preferably, be approximately the same length as a commercially available endotracheal tube exchanger, for example about 80-100 cm. In addition, referring to FIG. 3, markings can be provided on its wall indicating, for example in 5 cm increments, the length from its distal end to any given mark. This can allow the clinician to better gauge whether the distal tip is likely to be in the oropharynx, trachea, bronchus or beyond, and can therefore improve patient safety.

### EXAMPLE 4

Referring to FIG. 3, the subject invention, for example the devices disclosed in the previous examples, can have a hollow channel of sufficient caliber to provide adequate jet ventilation, suction and/or irrigation, if desired. Accordingly, if mucus plugs are visualized they can be suctioned and if secretions obstruct vision, the lens can be cleaned with irrigation through the hollow channel. Advantageously, ventilation can be provided through the hollow channel during the exchanging of endotracheal tubes.

The subject invention can also incorporate deflection, for example of about 0-60°, between about 1-10 cm from the distal tip, to allow a clinician to direct the subject device down either bronchus, towards a mucus plug, or away from the tracheal wall, simply by rotating the device along its length. This deflection may be incorporated into a device such that the distal tip of the subject scope conforms to the shape of an endotracheal tube while within the tube. When the distal end of the scope is advanced past the distal end of tube, the tip can assume a predefined deflection, for example due to memory in the stylet.

### EXAMPLE 5

The alternative embodiments described in this section, and as depicted in FIGS. 8-13, address the use of a small bore scope, such as an angioscope (Edwards Lifesciences Intramed 2.0 mm angioscope-Ref. 780020) in combination with a stylet adapted to work in detachable combination with the scope. The scope can be sterilized and re-used while the stylet can be disposable. By preferentially (but not necessarily) making use of the existing light sources and display means in the operating room (OR), the alternative embodiments described herein reduce OR clutter as well as the cost of video-intubation with an imaging stylet design.

The detachable stylets of the embodiments described herein are made to mate with the scope along most of the stylet's length, and will preferably allow shaping/bending of the stylet/scope combination with minimal forces generated that tend to separate the scope from the stylet, for example, by providing for relative longitudinal movement between the stylet and scope.

In some preferred alternative embodiments, the detachable stylets are made so as to possess one or more of the following characteristics: index the scope relative to the stylet so that the image is always properly oriented, e.g., not upside down; promote bending of the stylet in a preferred plane; require less material than a conventional circular cross-section stylet to achieve similar stiffness and bending moment in a stylet; further index the scope relative to the preferred bending plane of a stylet; control the relative longitudinal position of the scope tip relative to the stylet tip; and/or provide visual confirmation that the scope is properly oriented relative to the stylet.

In order to permit detachable mating of the stylet with the scope such that the stylet mates with the scope along most of its length, a channel is extruded along the external surface or sheath of a stylet into which a scope can be snapped, substantially along the length of the stylet. The degree to which the channel envelops the scope may vary. Such variations are exemplified in FIGS. 8,9,10,12, and 13.

Referring to FIG. 8, one can see a transverse cross-sectional view of a preferred embodiment of a malleable stylet/scope combination according to the subject invention with U-channel 212 into which a scope snaps. The stylet/scope combination is depicted within an endotrached tube 218, ready for insertion. Scope 210 in this representation has an imaging guide 214, two illumination fibers 215 and one suction/insufflation/irrigation lumen 216. Drawing is approximately to scale and the standard diameter stylet 211 is moved off-center with respect to the longitudinal axis within its sheath 217 to accommodate the U-channel 212. Note that the imaging guide lumen in the scope is shown as the lumen furthest away from the center of the stylet and closest to the internal wall of the endotracheal tube (ETT) 213, which may promote an undesirable fish-eye view. Ideally, the scope would be indexed such that the imaging guide is as close as possible to the center of the ETT when the scope/stylet combination is loaded into the ETT.

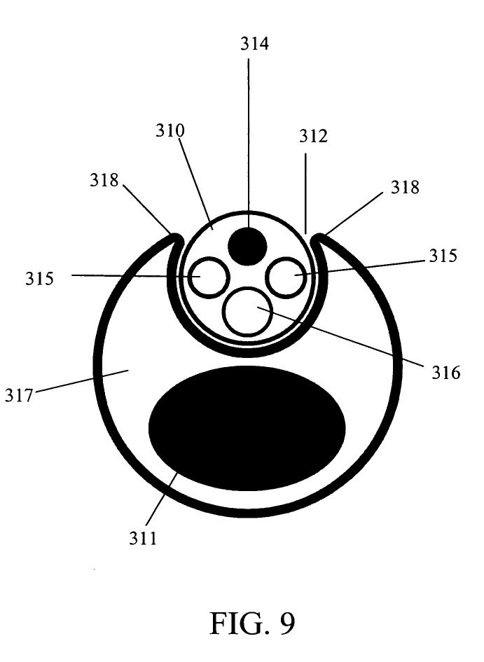

Turning now to FIG. 9, one sees a transverse cross-sectional view of yet another preferred embodiment of a stylet/scope combination according to the subject invention with C-channel 312 into which a scope 310 snaps. Scope 310 in this representation has an imaging guide 314, two illumination fibers 315 and one suction/insufflation/irrigation lumen 316. Drawing is approximately to scale. The stylet 311 transverse cross-section is made elliptical to accommodate the deeper C-channel 312 and is moved off-center with respect to the longitudinal axis within its sheath to accommodate the C-channel 312. C-channels where the two edges 318 of the channel almost meet are also contemplated, are within the scope of the subject invention, and are readily envisioned by the ordinary artisan in view of the teachings herein.

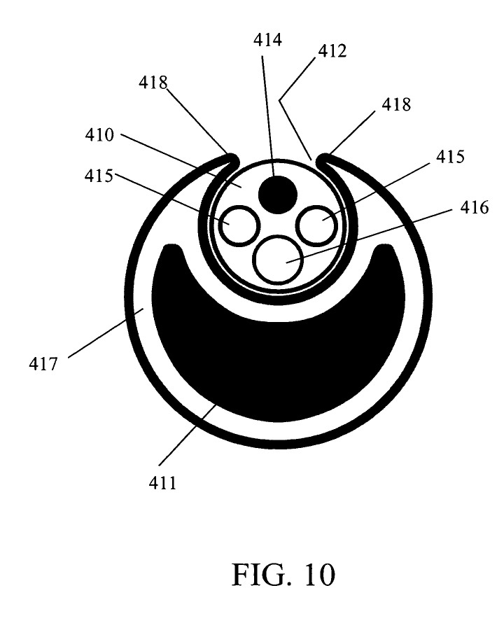

Turning now to FIG. 10, one sees a transverse cross-sectional view of yet another embodiment of a stylet/scope combination according to the subject invention with C-channel 412 into which a scope 410 snaps. Scope 410 in this representation has an imaging guide 414, two illumination fibers 415 and one suction/insufflation/irrigation lumen 416. Drawing is approximately to scale. The stylet 411 transverse cross-section is made crescent-shaped to accommodate a deeper C-channel 412 and is moved off-center with respect to the longitudinal axis within its sheath 417 to accommodate the C-channel 412. C-channels where the two edges 418 of the channel almost meet are also contemplated, are within the scope of the subject invention, and are readily envisioned by the ordinary artisan in view of the teachings herein.

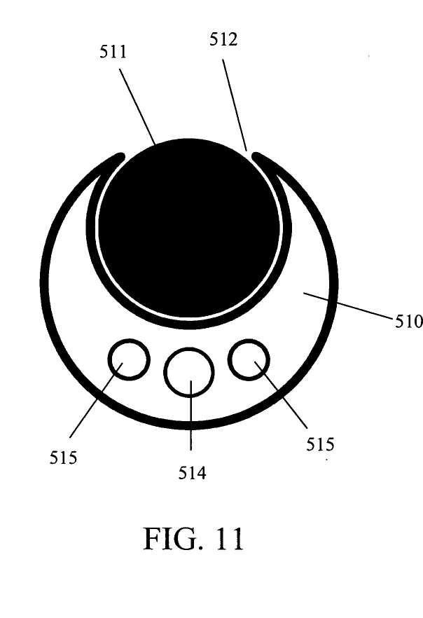

FIG. 11 shows an embodiment when the scope is made with an extruded or molded channel 512. This transverse cross-sectional view shows a stylet/scope combination according to the subject invention with C-channel 512 into which a stylet 511 snaps. Scope 510 in this representation has an imaging guide and two illumination fibers 515. Drawing is approximately to scale. The stylet 511 transverse cross-section and diameter as depicted, although not necessarily required by this embodiment, are those of a standard stylet.

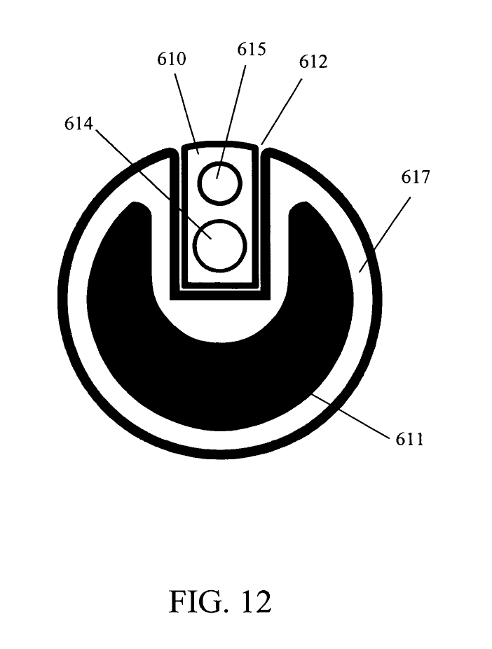

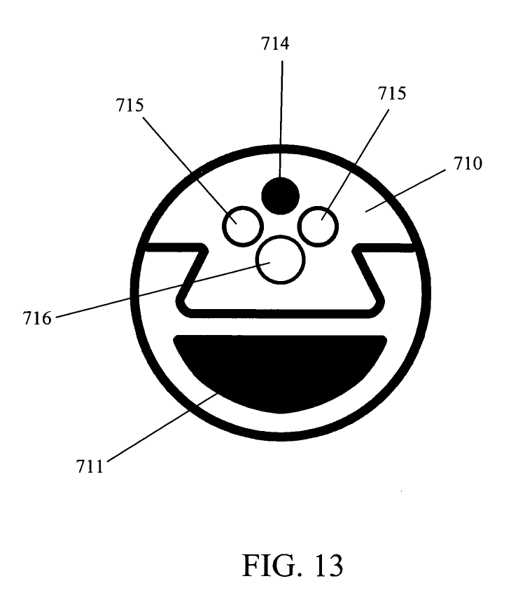

FIG. 12 is a transverse cross-sectional view of yet another embodiment of a stylet/scope embodiment according to the subject invention with a slot-channel 612 in sheath 617 into which a scope 610 snaps. Scope 610 in this representation has an imaging guide 614 and an illumination fiber 615. Drawing is approximately to scale. The shape of the scope provides indexing of the scope relative to the stylet 611. FIG. 13 a transverse cross-sectional view of still another embodiment of a stylet/scope combination according to the subject invention that provides positive indexing of the scope 710 relative to the stylet. Scope 710 is depicted with image guide 714, two illumination fibers 715, and lumen 716.

In order to allow more efficient shaping/bending of the stylet/scope combination, clearance is optionally and optimally provided between the surfaces of the scope and stylet where they engage so that relative longitudinal movement is facilitated while still retaining the scope within the scope/stylet combination.

Preferred embodiments of the stylet/scope combination which index the scope relative to the stylet are depicted in FIGS. 12 and 13. These embodiments provide mating cross-sections that prevent rotation of the scope relative to the stylet or vice versa. Embodiments which promote bending of the stylet in a preferred plane can readily be constructed by techniques known in the art such that cross-section of the stylet is like a channel beam, being more rigid in certain bending planes than others. Similarly, these construction techniques are easily and routinely applied by the ordinary artisan in view of these teachings to require less material than conventional stylet to achieve similar stiffness and bending moment, for example, by moving the material to the areas where it contributes most to stiffness.

Still other preferred embodiments further index the scope relative to the preferred bending plane of a stylet in the following manner. The cross-section of the stylet dictates the preferred bending plane. An indexed channel in the stylet or scope can also act as a visual cue to the preferred bending plane. An indexed channel thus not only indexes the scope relative to the stylet, but also relative to the bend that is placed in the stylet so that the image is always oriented as desired.

The relative longitudinal positions of the scope tip and the stylet tip to each other can be controlled in preferred embodiments. For example, the distal end of the channel can have a clear plate to prevent the scope protruding past the stylet tip or vice versa. Alternatively, or additionally, matching patterns of circumferential lines can be placed on the scope and stylet, which line up if the scope and stylet tips are properly aligned. Yet another option is to have a protruding tag on the scope that matches a corresponding slot in the stylet's channel (or vice versa, where the channel is in the scope, such as depicted in FIG. 11), when the scope and stylet tips are properly aligned. Similarly, to provide visual confirmation that the scope is properly oriented relative to the stylet, a longitudinal stripe on the scope that can be seen via the slot in the channel provides visual confirmation that the scope is properly indexed. The stripe can also help ensure that the scope is correctly oriented relative to its attachment (appropriately marked) with a viewing or imaging device such as a camera or charge coupled device (CCD).

### EXAMPLE 6

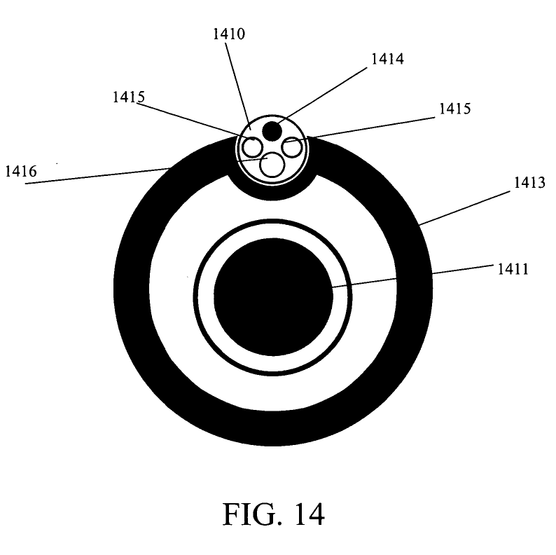

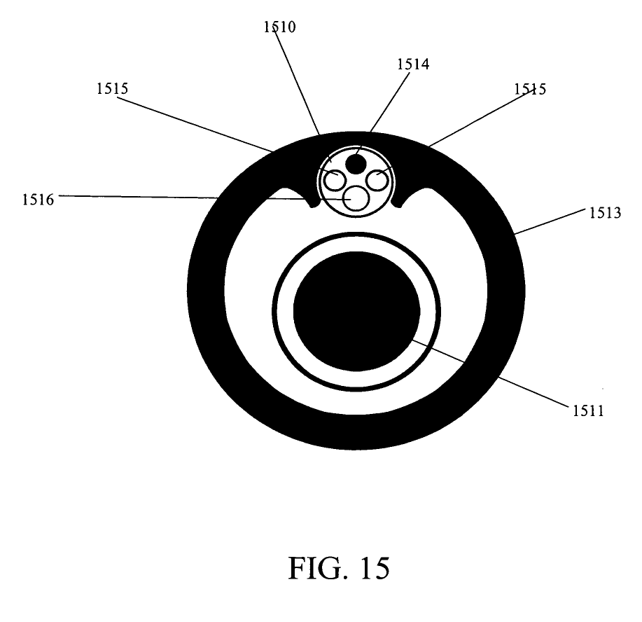

Still further preferred embodiments are depicted in FIGS. 14 and 15. Embodiments such as these are “combitubes” having channels located on the tube itself, either on the inside or the outside surface of the tube, and running substantially along the length of the tube. As will be readily apparent to one of ordinary skill in the art, such channels could be incorporated not only into endotracheal tubes, but other airway devices such as laryngeal mask airways (LMAs), intubating LMAs, and cuffed oropharyngeal airways (COPAs) and even any other intubating devices such as, but not limited to , gastric tubes, drainage tubes, chest tubes, urethral tubes, or any other tubes or catheters designed to be inserted into a human or other animal. Where insertion of the tube is desired, and where direct vision of insertion is also desired (as opposed to blind insertion), or where visual confirmation of placement and/or location is desired, intubation devices of the subject invention are advantageous. Referring to FIG. 14, there is presented a transverse cross-sectional view of an intubation device wherein the device is represented by an endotracheal tube 1413 having a channel with a C-shaped cross-section that has been formed along the length of the outside of the tube. As will be clear to those of ordinary skill in the art, although the cross-section of the channel depicted in FIG. 14 appears C-shaped, it could be just as easily be U-shaped, or any other shape, such as indexed shapes described and exemplified herein above, which permit engagement of a scope and/or stylet with the tube. FIG. 14 depicts scope 1410 having an imaging guide 1414, two rumination fibers 1415, and lumen 1416. A standard stylet 1411 is represented within tube 1413, however, as will be clear to the ordinary artisan in view of the teachings herein, a scope/stylet combination such as, for example, those depicted in FIGS. 8-13, or indeed any of scope/stylet combinations disclosed herein, could readily replace scope 1410 in the external channel, thereby eliminating the need for use of a standard stylet.

FIG. 15 depicts a transverse cross-sectional view of an intubation device according to the subject invention represented by an endotracheal tube 1513 having the longitudinal channel on the inside surface of the device. Scope 1510 is depicted mounted in a channel having a C-shape cross-section, but as with other embodiments described herein, the cross-sectional shape of the channel could be U-shaped, or any of the other shapes disclosed herein, including variations thereof which will be readily apparent to one of ordinary skill in the art, such that engagement of a scope and/or stylet with the tube is maintained once the scope and/or stylet is inserted into place. Scope 1510 includes imaging guide 1514, to illumination fibers 1515 and lumen 1516, as well as a standard stylet 1511. Although a standard stylet 1511 is depicted, as will be clear to the ordinary artisan in view of the teachings herein, a scope/stylet combination such as, for example, those depicted in FIGS. 8-13, or indeed any of scope/stylet combinations disclosed herein, could readily replace scope 1510 in the internal channel, thereby eliminating the need for use of a standard stylet.

It should be understood that the examples and embodiments described herein are for illustrative purposes only and that various modifications or changes in light thereof will be suggested to persons skilled in the art and are to be included within the spirit and purview of this application and the scope of the appended claims.

1. A device for intubating a human or other animal, comprising a tube and a channel formed in the wall of said tube, said channel running substantially parallel to the longitudinal axis of said tube.

2. An intubating device according to claim 1, wherein said tube is selected from the group consisting of endotracheal tubes, laryngeal mask airways, intubating laryngeal mask airways, cuffed oropharyngeal airways, gastric tubes, chest tubes, drainage tubes, and urethral tubes.

3. An intubating device according to claim 2, wherein said tube is selected from the group consisting of endotracheal tubes, laryngeal mask airways, intubating laryngeal mask airways, and cuffed oropharyngeal airways.

4. An intubating device according to claim 3, wherein said tube is an endotracheal tube.

5. An intubating device according to claim 1, wherein said channel is on the interior wall of said tube.

6. An intubating device according to claim 2, wherein said channel is on the interior wall of said tube.

7. An intubating device according to claim 3, wherein said channel is on the interior wall of said tube.

8. An intubating device according to claim 4, wherein said channel is on the interior wall of said tube.

9. An intubating device according to claim 4, wherein said channel is on the exterior wall of said tube.

10. An intubating device according to claim 2, wherein said channel is on the exterior wall of said tube.

11. An intubating device according to claim 3, wherein said channel is on the exterior wall of said tube.

12. An intubating device according to claim 4, wherein said channel is on the exterior wall of said tube.

13. An intubating device according to claim 5, further comprising an image guide disposed in said channel.

14. An intubating device according to claim 6, further comprising an image guide disposed in said channel.

15. An intubating device according to claim 7, further comprising an image guide disposed in said channel.

16. An intubating device according to claim 8, further comprising an image guide disposed in said channel.

17. An intubating device according to claim 9, further comprising an image guide disposed in said channel.

18. An intubating device according to claim 10, further comprising an image guide disposed in said channel.

19. An intubating device according to claim 11, further comprising an image guide disposed in said channel.

20. An intubating device according to claim 12, further comprising an image guide disposed in said channel.
